(19) United States
(12) Patent Application Publication
(10) Pub. No.: US 2021/0076813 A1
(43) Pub. Date: Mar. 18, 2021

(54) PLASMA TREATMENT DEVICE

(71) Applicant: CINOGY GMBH, Duderstadt (DE)

(72) Inventors: Dirk WANDKE, Heilbad Heiligenstadt (DE); Mirko HAHNL, Berlingerode (DE); Karl-Otto STORCK, Duderstadt (DE); Leonhard TRUTWIG, Duderstadt (DE); Melanie RICKE, Katlenburg-Lindau (DE)

(21) Appl. No.: 16/646,012

(22) PCT Filed: Sep. 3, 2018
(86) PCT No.: PCT/DE2018/100748

§ 371 Date: Dec. 4, 2020

(30) Foreign Application Priority Data
Sep. 11, 2017 (DE) 10 2017 120 902.6

(51) Int. Cl. A46B 15/00; A61N 1/44; A61C 17/22; A61C 15/00; A61L 2/00; A61L 2/26; H05H 1/24; A46B 9/04

(52) U.S. Cl. CPC A46B 15/0024; A61N 1/44; A61C 17/228; A61C 15/00; A61L 2/0011; H05H 2001/2418; H05H 1/2406; A46B 9/045; A61L 2202/11; A61L 2202/16; A61L 2202/17; A61L 2/26

(57) ABSTRACT

A plasma treatment device configured for a treatment within the oral cavity of a living organism using a dielectric barrier plasma, comprising a high-voltage stage (5, 6) arranged in a housing and serving for generating high-voltage signals required for generating the plasma, and comprising an electrode arrangement (10), which is connected to the high-voltage stage and is covered by a dielectric (9) for forming a dielectric barrier plasma, enables various treatments in the oral cavity of the living organism in a simple manner by virtue of the fact that the housing has a handle part (1), to which a head part (2) is secured in a releasable and exchangeable manner by means of a mechanical connection arrangement, in that the handle part (1) contains all stages required for generating the high-voltage signals and a contact arrangement, at which the high-voltage signals are present, in that the head part (2) is embodied in a shape specific to a specific treatment and has a corresponding shaped electrode arrangement (10), which is surrounded completely by the dielectric (9) and has a terminal (14), which makes contact with the contact arrangement of the handle part (1) when the head part (2) is secured to the handle part (1) in a manner effected by means of the mechanical connection arrangement.

The invention relates to a plasma treatment device, configured for a treatment within the oral cavity of a living organism using a dielectric barrier plasma, comprising a high-voltage stage arranged in a housing and serving for generating high-voltage signals required for generating the plasma, and comprising an electrode arrangement, which is connected to the high-voltage stage and is covered by a dielectric for forming a dielectric barrier plasma.

The treatment of surfaces of the skin, including wound areas, using a dielectric barrier plasma is known and attracting increasing attention. The plasma treatment is intended firstly to reduce germs at the surface of the skin or wound and secondly to stimulate the microcirculation of the tissue. This promotes wound healing, for example. Healthy skin can be cosmetically enhanced by the treatment. Furthermore, it is possible to combine the plasma treatment with the addition of healing or cosmetic care substances since the plasma treatment promotes the absorption of these substances by the skin.

When a dielectric barrier plasma is formed, a direct current flow is prevented by a dielectric barrier layer, with the result that an arc that would burn the skin or tissue cannot arise.

DE 103 24 926 B3 discloses, in principle, carrying out the plasma treatment using a dielectric barrier plasma on living cells. One example is also a tooth treatment. This makes use of a cylindrical, rod-shaped electrode, which is surrounded by a dielectric and generates a plasma field at its tip, in which plasma field high-voltage pulses are fed to the electrode and the body having the cells to be treated functions as a counter electrode. In this case, care is taken to ensure that a small plasma field arises at the tip of the electrode. A rod-shaped electrode having a circular-cylindrical lateral surface formed by the dielectric can be used for the treatment of a larger surface. An approximately linear plasma field arises in this case. In order to treat an area, the electrode is rolled over the area to be treated. The concept of sweeping over a larger area by rolling a spherical electrode or a roll-shaped electrode over it is furthermore the subject matter of DE 10 2013 019 058 A1 and EP 2 946 641 B1.

WO 2011/076193 A1 furthermore discloses configuring a flexible electrode for an areal treatment of a surface of skin, with which uniformly a larger area only the emplacement of the electrode arrangement is effected. In this case, the dielectric enclosing the live electrode bears directly against the surface of the skin on a treatment side, but is structured on this side such that between bearing points or lines there are air spaces in which the plasma can form.

All known plasma treatment devices are thus designed for punctiform, linear or areal surface treatment. This also holds true insofar as the treatment of teeth in the oral cavity has been taken into consideration in accordance with DE 103 24 926 B3. For this purpose, a plasma field having a very small extent is provided and can be used to sweep over the surface of a tooth in order to treat a caries attack, for example.

The present invention is based on the object of configuring a plasma treatment device provided for treatment within the oral cavity of a living organism such that a diverse and improved treatment is possible.

In order to achieve this object, according to the invention, a plasma treatment device of the type mentioned in the introduction is characterized in that the housing has a handle part, to which a head part is secured in a releasable and exchangeable manner by means of a mechanical connection arrangement, in that the handle part contains all stages required for generating the high-voltage signals and a contact arrangement, at which the high-voltage signals are present, in that the head part is embodied in a shape specific to a specific treatment and has a corresponding shaped electrode arrangement, which is surrounded completely by the dielectric and has a terminal, which makes contact with the contact arrangement of the handle part when the head part is secured to the handle part in a manner effected by means of the mechanical connection arrangement.

The plasma treatment device according to the invention has the special feature that the electrode arrangement is specifically shaped for a treatment within the oral cavity. The plasma treatment of teeth, gingiva and tongue has been possible only in a punctiform fashion hitherto. By virtue of the present invention, the head part with the electrode arrangement is shaped such that specific treatments not possible hitherto can actually be carried out or improved. Furthermore, what is achievable by virtue of the specifically shaped head parts is that for example all teeth of the maxilla or of the mandible can be treated jointly during the same treatment step. The exchangeability according to the invention of the head part on account of the releasable connection between head part and handle part furthermore makes it possible to carry out a comprehensive treatment within the oral cavity successively using the same handle part and merely an exchanged head part. By way of example, it is possible to treat firstly the teeth of the maxilla and/or of the mandible and then the interdental spaces using a different head part. In another exemplary embodiment, the tooth surfaces can be treated using a head part, while a hole made on account of a caries attack is able to be treated using a head part embodied in the shape of a needle, which can be introduced into the hole.

Therefore, the subject matter of the invention is furthermore a plasma treatment set consisting of a handle part and at least two differently shaped head parts, each of which is connectable to the handle part in a releasable manner.

In one embodiment of the invention, the head part is shaped in the manner of a dental splint having a base wall and two side walls, which makes it possible to reach around the teeth of a series of teeth of the living organism, wherein the shaped electrode arrangement is arranged areally in the bottom wall. In this embodiment, the teeth of a series of teeth (maxilla or mandible) are able to be treated jointly in the same treatment step. The shaping of the head part in the manner of a dental splint ensures a correct positioning of the electrode arrangement.

In another modified embodiment the head part is embodied with a planar area in a U-shape corresponding to the shape of a series of teeth. This head part can be used to treat in particular the occlusal surfaces of the teeth of the series of teeth.

In another embodiment, the head part is embodied as a thin flat part for insertion into an interdental space. This is used to treat in particular the tooth edges of adjacent teeth facing one another which is advisable particularly in the case of a caries attack in the interdental space. It is furthermore possible for the head part to be embodied in the form of a toothbrush, which is optionally electrically movable. It is thereby possible to combine the plasma treatment with mechanical cleaning of the teeth.

In another embodiment, the head part has a frame shape having a central opening, into which a tongue of the living organism can project, wherein the electrode arrangement extends over the entire frame shape, that is to say is likewise embodied in the shape of a frame. This head part is suitable in particular for removing a bacterial coating on the tongue, wherein the frame-shaped head part is drawn solely over the length of the tongue with a proximal transverse ridge.

In a further embodiment, the head part is embodied in the shape of a thin rod of a size allowing it to be introduced into a hole made in a tooth. Since it the electrode arrangement likewise extends in this shape of a thin rod, a hole made in a tooth can be treated using such a head part using a dielectric barrier plasma.

In a further embodiment of the invention, the head part is embodied with an areal base section having a width and a length, from which rise over the length a plurality of ridges extending over the width and configured for being introduced into interdental spaces. The head part is similar to the bristle arrangement of a toothbrush, wherein the ridges preferably consist of highly flexible dielectric plastic. Silicone, in particular, is suitable for this. The ridges can then be pressed at least partly into interdental spaces, even if the distance between the ridges does not completely correspond to the center-to-center distance between the interdental spaces. In this case, the electrode arrangement is preferably embodied integrally for projecting into the ridges of the dielectric. In this case, besides the ridges, it is also possible for bristles to be positioned, by means of which the plasma treatment can be combined with mechanical cleaning.

In all embodiments, the electrode arrangement can be embodied as one electrode, to which the high-voltage pulses are applied, wherein the parts of the oral cavity that are to be treated act as a counter electrode. However, the electrode arrangement can also consist of two electrodes insulated from one another. In this embodiment, the two electrodes can both be supplied with high-voltage pulses that are identical, but in antiphase. This results in a zero field at some distance. That part of the oral cavity which is to be treated serves as a counter electrode here as well.

In the case where the electrode arrangement is embodied with two electrodes, it is furthermore possible to connect one of the electrodes to the high-voltage signals and the other to a reference potential. In this case, the plasma field does not form between the electrode arrangement and that part of the oral cavity which is to be treated as counter electrode, but rather between the two electrodes. Since, as is known, the field lines extend arcuately at the edge of the electrodes, a surface plasma can be generated in this arrangement, although it is formed less efficiently.

The invention will be explained in greater detail below on the basis of exemplary embodiments illustrated in the drawing, in which:

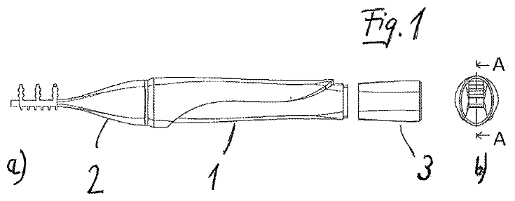

FIG. 1 shows illustrations of one exemplary embodiment with a first embodiment of a head part, which is able to be secured to the handle part of the plasma treatment device in a releasable manner;

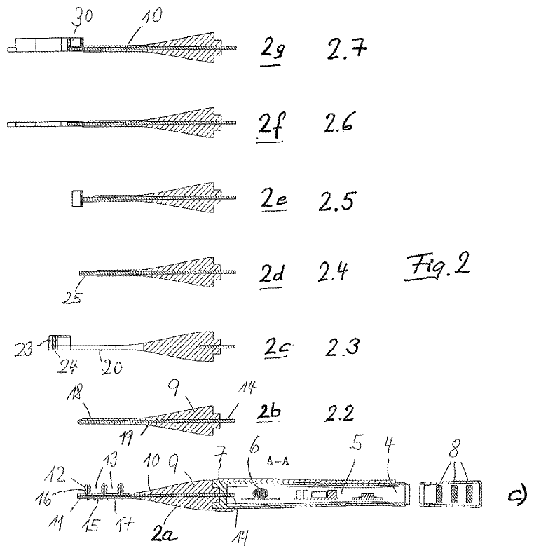

FIG. 2 shows sectional illustrations analogous to the sectional line A-A in FIG. 1b) for six further exemplary embodiments of head parts that are connectable to the handle part illustrated in FIG. 1;

FIG. 3 shows plan views of the top sides of the seven head parts illustrated in FIGS. 1 and 2;

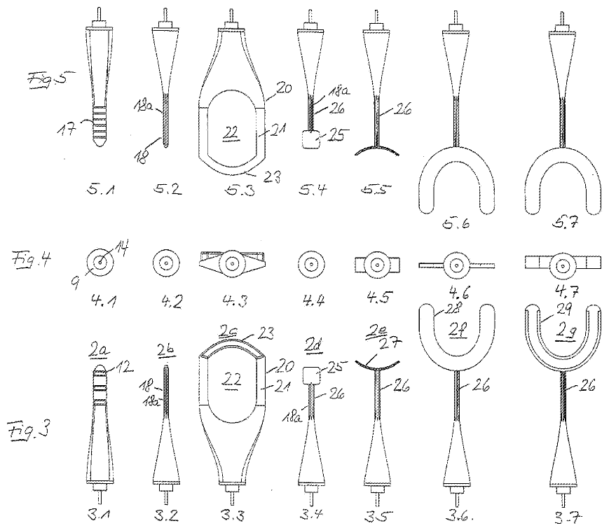

FIG. 4 shows rear views—as seen from the handle part—of the seven head parts in accordance with FIG. 3;

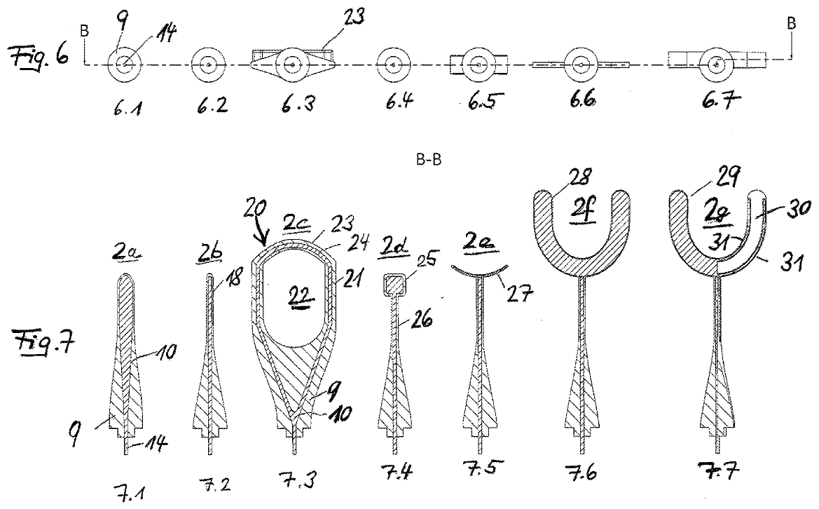

FIG. 5 shows views of the undersides of the head parts in accordance with FIG. 3;

FIG. 6 shows rear views in accordance with FIG. 4 with an illustration of a sectional line B-B;

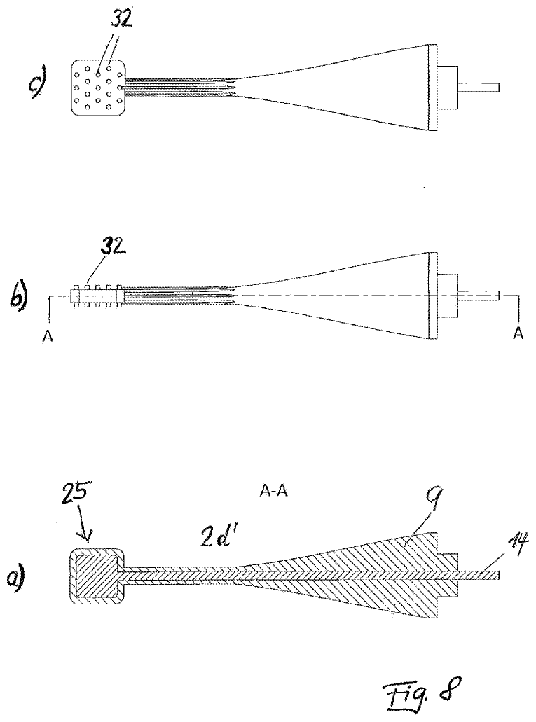

FIG. 7 shows horizontal sectional views through the seven embodiments of head parts along the sectional line B-B from FIG. 6;

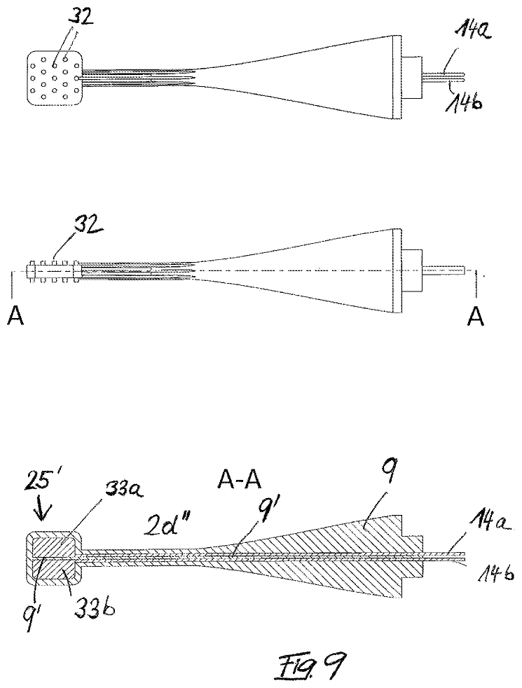

FIG. 8 shows a sectional illustration in accordance with FIG. 7, a side view in accordance with FIG. 1 and a plan view in accordance with FIG. 3 of a modified head part;

FIG. 9 shows illustrations in accordance with FIG. 8 for a further modified head part.

FIG. 1 shows a complete plasma treatment device in a side view (FIG. 1a)), as seen in an end view of the head part (FIG. 1b)) and in a vertical sectional view along the sectional line A-A from FIG. 1b) (FIG. 1c)). The illustrated exemplary embodiment of a plasma treatment device has a housing composed of a handle part 1, a head part 2 and a rear terminating part 3. The connections between handle part 1 and head part 2 and also between handle part 1 and rear terminating part 3 are preferably snap-action connections, but can also be embodied as screw connections, bayonet connections or the like. The handle part 1 has a slightly ergonomically shaped outer contour, which is intended to make it possible to securely grip the plasma treatment device. The hollow handle part 1 has an interior 4, in which an electronic controller 5 is situated, which generates AC voltage pulses from a DC voltage, said pulses being converted into high-voltage pulses by a coil arrangement 6. Controller 5 and coil arrangement 6 thus form a high-voltage stage 5, 6. The high-voltage pulses pass to a contact arrangement (not illustrated in more specific detail) in the form of a socket, which is inserted into an insulating terminating piece 7 at the end and via which the high-voltage pulses generated can be transmitted to the head part 2.

The rear terminating part 3 encloses three batteries 8, which are readily accessible and exchangeable by removal of the rear terminating part 3. The exemplary embodiment illustrated in FIG. 1 thus represents a handheld device which manages without supply cables and is therefore conveniently handleable. Within the scope of the invention, however, it is also conceivable to supply the device with a power feed via a cable, wherein the power supply can be a DC voltage supply or an AC voltage supply, for example from the public electricity grid. In the last-mentioned case, the controller 5 expediently contains a rectifier and chopper stage.

In the exemplary embodiment illustrated in FIG. 1, the head part 2 is embodied as a specifically shaped head part 2a having an integral dielectric 9, which shields an electrode arrangement 10 against touch when the head part 2a is properly connected to the handle part 1. The dielectric 9 is shaped for the head part 2a such that it tapers proceeding from an approximately cylindrical shape of the handle part 1 and transitions to an areal base section 11, from which three ridges 12 extend upward and form interspaces 13 between them. At the end facing the handle part 1, the electrode arrangement forms a conductive terminal 14 in the form of a terminal which projects from the dielectric 9 and which is pluggable into the abovementioned socket in the terminating piece 7 of the handle part 1 and thus establishes the contact with the coil arrangement 6. The electrode arrangement 10, proceeding from the terminal 14, extends into the areal base section 11 and forms an areal electrode section 15 there. Ridge attachments 16 are integrally connected to said areal electrode section 15, said attachments extending centrally into the ridges 12 and being enclosed on all sides by the dielectric of the ridges 12. The complicated shape of the electrode arrangement 10 can be embodied integrally if the electrode arrangement is formed from a moldable plastic to which electrically conductive additives, preferably in the form of particles, are added. A suitable moldable material is a silicone that binds in a materially bonded manner with an electrically insulating silicone used as dielectric.

The ridges 12 serve for positioning the head part 2a in interdental spaces into which they can be pressed—at least partly. The ridges 12 consist of a soft-flexible material and can therefore penetrate into interdental spaces even if the width of the tooth does not completely correspond to the width of the interspace 13 between the ridges 12. Since the electrode arrangement 10 with the electrode section 15 and the ridge attachments 16 can surround the tooth from three sides, the best possible complete plasma treatment of the teeth situated between the ridges 12 is made possible.

As is illustrated in FIG. 1, the dielectric in the region of the ridges 12 can be embodied with a sawtooth-shaped profile, for example, in order to facilitate penetration into interdental spaces.

On the rear side of the base section 11 facing away from the ridges 12, it is possible to provide, transversely with respect to the longitudinal direction of the head part 2a, reinforcing ridges 17 or weakening grooves, which increase the elasticity of the dielectric 9 in the region of the base section 11.

The first exemplary embodiment of a head part 2a is illustrated in various views in FIGS. 3 to 7 and in a further sectional illustration in FIGS. 3.1 to 7.1.

A head part 2b in a second embodiment is illustrated in FIGS. 2 to 7 respectively in FIGS. 2.2 to 7.2. The dielectric 9 extends in a manner proceeding from the terminal piece with respect to the handle part 1 via a taper into a thin rod-shaped end 18, the diameter of which is less than 1 mm and which is provided with longitudinal grooves 18a, which firstly bring about mechanical reinforcement and secondly, in the course of their bearing, for example against a tooth, bound air spaces in which the plasma can arise. The electrode arrangement 10 extends from the terminal 14 in the shape of a pin into the rod-shaped end 18 and forms a wire-like electrode core 19 for the rod-shaped end 18. At the end face, the electrode core 19 is of course covered by the dielectric, here with a rounded end.

A third embodiment of a head part 2c is illustrated in FIGS. 2 to 7 with FIGS. 2.3 to 7.3. In this case, the dielectric 9 tapers to form a flat piece 20, which forms a frame 21 having a central opening 22. The opening is closed by an elevated terminating ridge 23 at the distal end with respect to the handle part 1. The opening 22 is dimensioned such that it can receive at least part of the tongue. The terminating ridge 23 forms a scraping edge by which the head part 2c can be drawn over the tongue in order to remove a bacterial coating of the tongue, for example. Here, too, the electrode arrangement 10 forms the terminal 14 and bifurcates within the dielectric 9 in order to enclose the opening 22 in the plane of the flat piece 20 centrally at the level of the dielectric 9, wherein in the region of the terminating ridge 23 the electrode arrangement 10 is also shaped into an elevated ridge 24. The head part 2 is thus shaped specifically for the treatment of the tongue.

In a fourth embodiment, a head part 2d is embodied as a flat planar end piece 25 situated at a narrow and therefore highly elastic rod-shaped transition piece 26 likewise with longitudinal grooves 18a. This head part is specifically suitable for the treatment of attacked interdental spaces since the areal electrode in the end piece 25 gives rise to a concentrated plasma field on both sides of the end piece, said plasma field acting on the adjacent teeth when the head part 2d is introduced into an interdental space.

In accordance with the fifth embodiment illustrated in FIGS. 2 to 7 in FIGS. 2.5 to 7.5, the head part 2e likewise has a rod-shaped transition piece 26, adjacent to which is an end piece 27 in the shape of an arc segment. The end piece is perpendicular to the plane formed by the electrode arrangement 10 and is suitable, in particular, for regionally treating outer regions of the jaw, in particular at the transition between gingiva and tooth.

In a sixth embodiment, illustrated in FIGS. 2.6 to 7.6 of FIGS. 2 to 7, the head part 2f, which for the rest is embodied in the same way as in the fourth and fifth exemplary embodiments, has a U-shaped end piece that forms a U-shaped strip in the plane of the electrode arrangement 10. The head part 2f is therefore suitable in particular for the treatment of the occlusal surfaces and edges of maxilla and mandible when the teeth bear against the U-shaped end piece at the top side and the underside.

In a seventh exemplary embodiment, illustrated in FIGS. 2.7 to 7.7 of FIGS. 2 to 7, the head part 2g has an end piece 29 in the manner of a dental splint, such that the dielectric 9 forms an upwardly open channel having a base wall 30 and two side walls 31. The electrode arrangement 10 extends at least over the base wall, but can also extend into the side walls. This head part 2g is suitable for the treatment of the entire teeth of a maxilla or mandible.

FIG. 8 shows in an enlarged illustration a horizontal sectional view (FIG. 8a), a side view (FIG. 8b) and a plan view (FIG. 8c) of a modified head part 2d′. The modified head part 2d′ differs from the head part 2d in FIGS. 2.4 to 7.4 in that the dielectric 9 at the surface of the flat planar end piece 25 is embodied with small pimples 32, by which the flat planar end piece 25 can bear against a body part to be treated, wherein between the pimples 32 there are air spaces in which the plasma can form. The pimples 32 form merely one exemplary embodiment of a structured surface, which can also be realized at the other head parts 2, also in the form of a grid-like structure, a pore structure, etc. An identical function is fulfilled by the reinforcing ribs 17 at the head part 2a and the longitudinal grooves 18a at the embodiments of the head parts 2b, 2d, 2e, 2f and 2g. It is evident that the pimples 32 are not tied to specific arrangement, and so the illustration in FIG. 8c serves merely for illustrating one example.

FIG. 9 shows views as in FIG. 8 for a further modified head part 2d″. While the embodiment in accordance with FIG. 8 has a single electrode in the flat piece 25, the head part 2d″ contains two electrode areas 33a, 33b lying next to one another in the flat planar end piece 25′, said electrode areas being able to be connected to the required high voltage via terminal lines 14a, 14b running parallel in the dielectric 9. The two terminal lines 14a, 14b, just like the electrode areas 33a, 33b, are insulated from one another by a central dielectric strip 9′.

Preferably, the two electrode areas 33a and 33b are supplied with identical high-voltage pulse signals, which are in each case of opposite polarity with respect to one another, such that the fields formed are already extinguished at a small distance and the plasma arises only at close range. For the rest, the head part 2d″ corresponds externally to the head part 2d′ and is preferably likewise provided with pimples 32 or some other structuring of the dielectric 9.

The exemplary embodiments illustrated clarify that with one device, by exchanging the head parts 2, it is possible to carry out various plasma treatments in the oral cavity because the head parts 2 have shapes adapted to the respective treatment measure.

1. A plasma treatment device configured for a treatment within the oral cavity of a living organism using a dielectric barrier plasma, comprising
a housing,
a high-voltage stage arranged in the housing for generating high-voltage signals required for generating the plasma,
an electrode arrangement which is connected to the high-voltage stage and is covered by a dielectric for forming a dielectric barrier plasma,
wherein the housing has a handle part, to which a head part is secured in a releasable and exchangeable manner by a mechanical connection arrangement,
wherein the handle part contains all stages required for generating the high-voltage signals and a contact arrangement at which the high-voltage signals are present, and
wherein the head part is embodied in a shape specific to a specific treatment and has a corresponding shaped electrode arrangement which is surrounded completely by the dielectric and has a terminal which makes contact with the contact arrangement of the handle part when the head part is secured to the handle part in a manner effected by the mechanical connection arrangement.

2. The plasma treatment device according to claim 1, wherein the head part is shaped in the manner of a dental splint having a base wall and two side walls, which makes it possible to reach around the teeth of a series of teeth of the living organism, and wherein the shaped electrode arrangement is arranged at least areally in the bottom wall.

3. The plasma treatment device according to claim 1, wherein the head part comprises a planar area with a U-shaped end piece corresponding to the shape of a series of teeth.

4. The plasma treatment device according to claim 1, wherein the head part is a thin flat end piece for insertion into an interdental space.

5. The plasma treatment device according to claim 1, wherein the head part has a frame shape having a central opening, into which a tongue of the living organism can at least partly project, and wherein the electrode arrangement has a corresponding frame shape.

6. The plasma treatment device according to claim 1, wherein the head part has a shape of a thin rod of a size allowing it to be introduced into a hole made in a tooth.

7. The plasma treatment device according to claim 1, wherein the head part is embodied with an areal base section having a width and a length, from which rise over the length a plurality of ridges extending over the width and configured for being introduced into interdental spaces.

8. The plasma treatment device according to claim 7, wherein the electrode arrangement extends areally in the base section and integrally into the ridges.

9. The plasma treatment device according to claim 7, wherein the head part is a toothbrush.

10. A plasma treatment set, comprising
a plasma treatment device; and
at least two different head parts,
wherein the plasma treatment device comprises
a housing,
a high-voltage stage arranged in the housing for generating high-voltage signals required for generating the plasma, and
an electrode arrangement which is connected to the high-voltage stage and is covered by a dielectric for forming a dielectric barrier plasma,
wherein the housing has a handle part to which a head part of the at least two different head parts is securable in a releasable and exchangeable manner by a mechanical connection arrangement,
wherein the handle part contains all stages required for generating the high-voltage signals and a contact arrangement at which the high-voltage signals are present, and
wherein the head part is embodied in a shape specific to a specific treatment and has a corresponding shaped electrode arrangement which is surrounded completely by the dielectric and has a terminal which makes contact with the contact arrangement of the handle part when the head part is secured to the handle part in a manner effected by the mechanical connection arrangement.

11. The plasma treatment set according to claim 10, wherein the head part is shaped in the manner of a dental splint having a base wall and two side walls, which makes it possible to reach around the teeth of a series of teeth of the living organism, and wherein the shaped electrode arrangement is arranged at least areally in the bottom wall.

12. The plasma treatment set according to claim 10, wherein the head part comprises a planar area with a U-shaped end piece corresponding to the shape of a series of teeth.

13. The plasma treatment set according to claim 10, wherein the head part is a thin flat end piece for insertion into an interdental space.

14. The plasma treatment set according to claim 10, wherein the head part has a frame shape having a central opening into which a tongue of the living organism can at least partly project, and wherein the electrode arrangement has a corresponding frame shape.

15. The plasma treatment set according to claim 10, wherein the head part has a shape of a thin rod of a size allowing it to be introduced into a hole made in a tooth.

16. The plasma treatment set according to claim 10, wherein the head part is embodied with an areal base section having a width and a length, from which rise over the length a plurality of ridges extending over the width and configured for being introduced into interdental spaces.

17. The plasma treatment set according to claim 16, wherein the electrode arrangement extends areally in the base section and integrally into the ridges.
